(19) United States
(12) Patent Application Publication
(10) Pub. No.: US 2019/0365011 A1
(43) Pub. Date: Dec. 5, 2019

(54) VENTILATION DEVICES FOR ARC-PROTECTIVE GARMENTS

(71) Applicant: PAULSON MANUFACTURING CORPORATION, Temecula, CA (US)

(72) Inventor: Roy PAULSON, Murietta, CA (US)

(73) Assignee: PAULSON MANUFACTURING CORPORATION, Temecula, CA (US)

(21) Appl. No.: 15/993,248

(22) Filed: May 30, 2018

(51) Int. Cl. A42B 3/28; A42B 3/22; A61F 9/06

(52) U.S. Cl. CPC A42B 3/283; A42B 3/286; A61F 9/068; A42B 3/225

(57) ABSTRACT

A ventilation device for use in protective hoods. The ventilation device is formed with facing plates, each plate having a plurality of slots therethrough, the slots in one plate being in offset registration with the slots in the other plate, thereby creating a semi-tortuous path for air flow through the ventilation device. Alternatively, the ventilation device may be a single, integral element, or each plate may be made in two or more segments. The ventilation device may be mounted in an opening in a hood, and several ventilation devices may be mounted in several openings in the hood.

### FIELD OF INVENTION

The present apparatus relates generally to the field of safety devices which protect people from injury arising from electric-arc discharges and more particularly to protective clothing, such as hoods and shrouds, which may be used together with safely helmets and face shields, for example.

### BACKGROUND OF THE INVENTION

Electrical-arc-flash hazards are a known threat in some workplaces and must be addressed to protect people who may be exposed to such dangerous conditions. Electric-arcs or flashes can result from short circuits developing from poor electrical grounding, failure of insulation, or workers inadvertently contacting exposed electrical circuit elements with objects such as tools. Electric-arcs have extremely high temperatures and near explosive power, and the energy they radiate can result in serious or fatal injury. The energy of an arc-flash is very intense and of very short duration. Flame resistant protective gear must be durable and able to withstand temperatures that may be as high as 35,000° F. To protect workers from exposure to such arc-flash events, a number of protective safety devices have been developed. In particular, face shields employing generally transparent windows comprised of compositions which have the ability for the user of the shield to see the workspace and, at the same time, have the ability to substantially block harmful radiation, are available. These devices are designed to provide protection against the thermal, optical, and mechanical hazards generated by arc-flash events. The protective compositions are referred to as energy absorbing materials and are classified by their caloric ratings, that is, the level of energy they have been tested or certified for.

When situational conditions or regulations so require, protective hoods are employed to protect the user's neck and upper shoulder area from arc-flash damage. Lack of adequate ventilation is a typical consequence of the fact that hoods must be lightly sealed to the user's helmet, or face shield, or both, and envelope at least part of the user's upper body. Under typical working conditions, the upper body and head of the user, being closely confined, can become uncomfortably hot and humid, and subject to build-up of CO₂, due to inadequate provisions for air to easily exhaust from the hood. It has been difficult to ventilate such protective clothing because of the potential arc-flash energy that could penetrate the clothing through openings intended to provide ventilation. Fan systems have been proposed with such hoods and have had some success. They would likely be battery powered. Fans are likely to add unwanted weight and noise.

### SUMMARY OF EMBODIMENTS OF THE INVENTION

A purpose of the present concept is to maintain electromagnetic energy and infrared energy protection while providing ventilation for the user of protective face shields with arc-protective clothing, such as hoods or shrouds, jackets, bibs, or suits. The term “hood” will be used herein as a convenient means to refer to all such arc-protective clothing.

A generally flat or planar ventilation device or assembly is mounted in an opening in the protective clothing. The mounting is sufficiently sealed so that the protective function of the hood is not compromised by installing one or more such ventilation devices.

The ventilation device is generally comprised of two spaced facing plates, an inner plate and an outer plate. Slots in the inner vent plate are in offset registration with slots in the outer vent plate, thereby providing ventilation without degrading electromagnetic energy protection. The assembly of a paired inner vent plate with an outer vent plate may be mounted at any location, or more than one location, in a hood as are typically employed in the field of protective clothing. The typical preferred location is at the back of the neck of the hood as worn by the user.

The term “offset registration” means that there is no direct pass-through route through the ventilation assembly and that air, for example, must move in a zig-zag, or semi-tortuous, path to pass from the inside to the outside, or vice versa. This offset registration results in electrical are radiation being blocked and thereby allowing ventilation of heated air inside the hood while maintaining safety of the user from electric arc energy.

In an alternative embodiment, the ventilation device is formed of an outer plate having several parallel slow therethrough, and an inner baffle plate connected to and spaced from the outer slotted plate. The baffle plate is not formed with slots and it performs essentially the same function as the two facing plate embodiment with slots in offset registration.

While the device disclosed herein is particularly useful with arc-flash protective gear, it is also useful for any work environment where a hood is worn by the user for protection and heat builds up inside the hood.

### BRIEF DESCRIPTION OF THE DRAWING

The purposes, features, and advantages of the disclosed structure will be more readily perceived from the following detailed description, when read in conjunction with the accompanying drawing, wherein:

FIG. 1 is a plan view of an embodiment of the ventilation device of this invention;

FIG. 1A is a partial sectional view taken along cutting plane 1A-1A of FIG. 1:

FIG. 2 is an exploded perspective view of the ventilation device of FIG. 1 from one side;

FIG. 3 is an exploded perspective view of the ventilation device of FIG. 1 from the opposite side;

FIG. 4 is a plan view, similar to FIG. 1, of an alternative embodiment of the ventilation device of this invention;

FIG. 5 is an exploded perspective view of the ventilation device of FIG. 4;

FIGS. 6A-6E are plan views of alternative shapes of embodiments of the subject ventilation device;

FIG. 7 is a perspective view of a protective hood With a face shield of the prior art;

FIG. 8 is a perspective view of a shroud connected to a safely helmet and having a face shield of the prior art;

FIG. 9 is a somewhat schematic view of the back of a hood as it might appear with one ventilation device of FIG. 1 mounted therein;

FIG. 9A is a partial sectional view of the ventilation device and hood fabric of FIG. 9;

FIG. 10 is a partial perspective view of a segment of the back of a hood with a group of four ventilation devices of FIG. 1 mounted therein;

FIGS. 11A-11E are partial perspective views similar to FIG. 10 showing groupings of the ventilation devices of FIG. 6 mounted in the fabric of a hood;

FIGS. 12A-12C are schematic partial sectional views along the lines of FIG. 9A showing alternative mountings of embodiments of ventilation devices of the prior figures as they can be secured in openings in a hood fabric; and

FIGS. 12D-12F show an alternative embodiment of the ventilation devices of this invention as mounted in different ways in openings in a hood fabric.

### DETAILED DESCRIPTION OF THE PREFERRED EMBODIMENTS

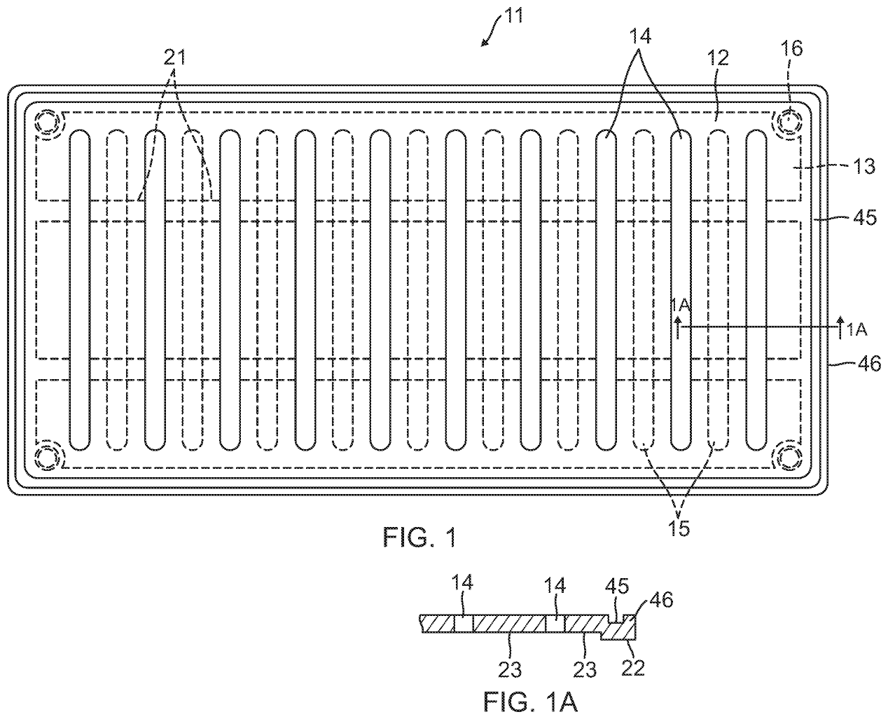

With reference now to the drawing, and more particularly to FIGS. 1-3, ventilation device 11 is preferably comprised of front plate 12 mounted to back plate 13. The terms “front” plate and “back” plate are used for convenience only and are not intended to designate a particular of those plates to be outside or inside with respect to the clothing items to which the ventilation device is intended to be mounted. Either, or both plates, could be made of more than one plate or as a unitary plate, as is deemed practical and appropriate for the purpose for which it is intended to be used. As further alternatives, the entire ventilation device 11 could be formed as a unitary product, which could be injection molded, 3-D printed, or made by some other process.

For purposes of this description, front and back plates 12 and 13 and device 11 will be discussed as having the parameters set forth below, no matter how the device is formed or assembled.

Plate 12 is formed with a plurality of parallel slots 14 therethrough. Plate 13 is formed with a plurality of parallel slots 15 therethrough. As shown, there are ten slots in plate 12 and nine slots in plate 13. There is no requirement that the number of slots be any particular number, only that they be offset with respect to each other when the plates are assembled in face-to-face relationship. Also, the number of slots through plate 12 may be the same as the number of slots in plate 13. That structural requirement will be discussed further below.

Plate 12 is shown formed with registration pins 16 near the periphery of each corner. Plate 13 is formed with similarly positioned matching holes 17. Those may be blind holes or through holes. Holes 17 are configured to match with pins 16 so that plates 12 and 13 can be secured together in fixed registration relationship. It is preferred that plates 12 and 13 be secured together to form a unitary device, as shown in FIG. 1.

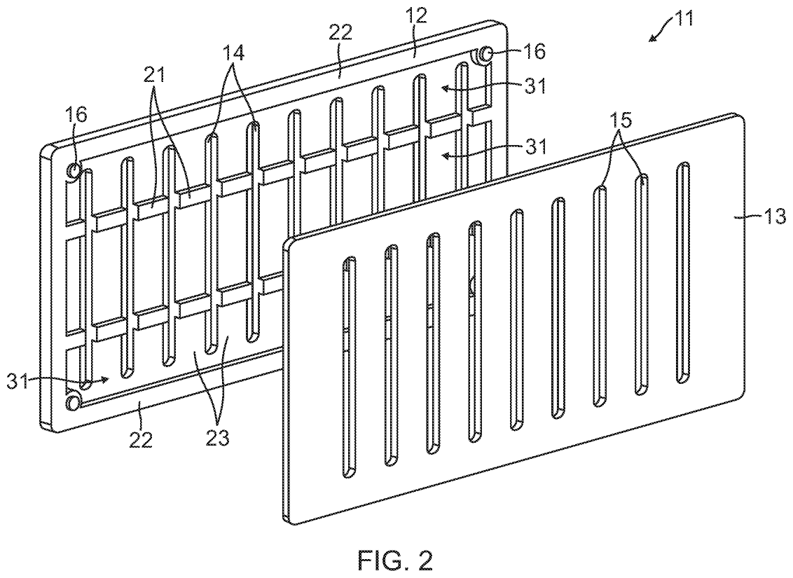

The side of plate 12 facing plate 13 is formed with stand-offs 21 which contact the inside surface of plate 13 and maintain air flow spaces 31 between the plates. As shown in FIG. 2, rim 22 and stand-offs 21 are all of the same thickness. As an alternative way of saying it, the rim and the stand-offs are in the same plane on the side of plate 12 facing plate 13. The remaining portion of the inside surface of plate 12, including lands 23, is depressed, or thinner than are rim 22 and stand-offs 21, thereby forming lateral air flow spares 31.

As can be seen in FIG. 1, when holes 17 in plate 13 are in registration with pins 16 in plate 12 and the two plates are mounted together, slots 14 and 15 are in offset registration, as previously stated. Air can flow in through slots 14, pass laterally in spaces 31 between the plates for a short distance between the inside surface of plate 13 and lands 23, and flow out through slots 15 in plate 13 in a semi-tortuous manner. Of course, flow could just as well be in the opposite direction. Because plate 13 is planar and rim 22 and stand-offs 21 are co-planar, air is generally confined to spaces 31 for lateral movement. However, it is not necessary that plates 12 and 13 be sealed together, but they would generally be secured together and the inside surface of plate 13 would be in contacting relation with rim 22 and stand-offs 21.

The space 31 between the inside surface of plate 13 and lands 23 of plate 12, in relation to the width of the slots, should be just enough to prevent any direct flow of are energy through device 11. One test to ensure that arc energy cannot pass directly through device 11 is to view the device normal to the slots and at a 45° angle from above the device. If there is no direct visibility through device 11 from that position, there would be no penetration of arc-flash energy therethrough. Stated another way, space 31 needs to be only sufficiently separated from the inside surface plate 13 to permit lateral air flow between plates 12 and 13 within the spaces 31.

Ventilation device 11 may be formed of known arc-energy resistant or absorbing material, such as rubber or a thermoplastic, having the same calorie rating as would a face protective shield or window and the hood used with the window. There may be several formulations of the constituents of which plates 12 and 13 are formed. Device 11 must be electrically non-conductive. Any which meet the protective, energy absorbing requirements, of the hood fabric and the protective window would normally be acceptable for these ventilation device plates.

Ventilation device 11 may be made from a thermoplastic elastomers or thermoplastic vulcanizate, such as SANTOPRENE, a trademark registration of Exxon Mobil Corporation. Alternative materials from which device 11 can be formed include EPDM rubber, silicone rubber, or neoprene rubber. There may be other high temperature resistant materials that are acceptable. As a further example, carbon black may be added to SANTOPRENE to result in an arc-energy blocking device. Other materials that are tuned to the electromagnetic spectrum of an arc-flash (200-3000 nm as an example), include organic dyes, nanoclays, and nanoparticles.

The structure of device 11 ensures that there is no direct, unimpeded fluid flow route from one side to the other, so the user is protected from potential arc-flash injury. At the same time, ventilation air can flow through device 11 as previously described, providing much needed ventilation for the user when wearing a ventilation device equipped hood.

Plate 12 is formed with narrow peripheral groove 45 (FIGS. 1, 3, and 4) in rim 22, just inside edge 46. This is used in securing ventilation device 11 to clothing, as will be discussed below. This is shown clearly in the cross-sectional view of FIG. 1A.

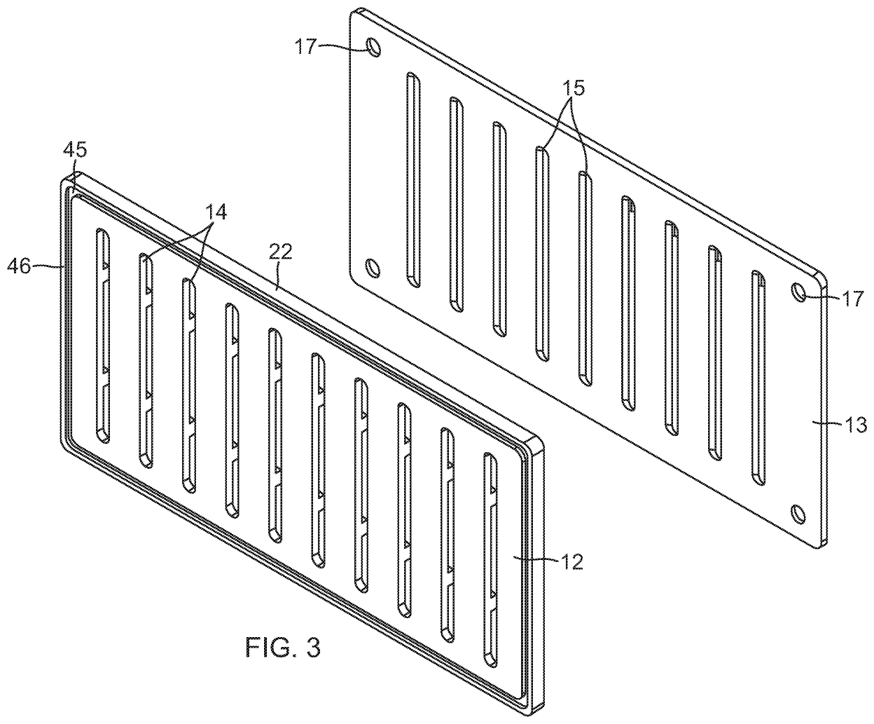

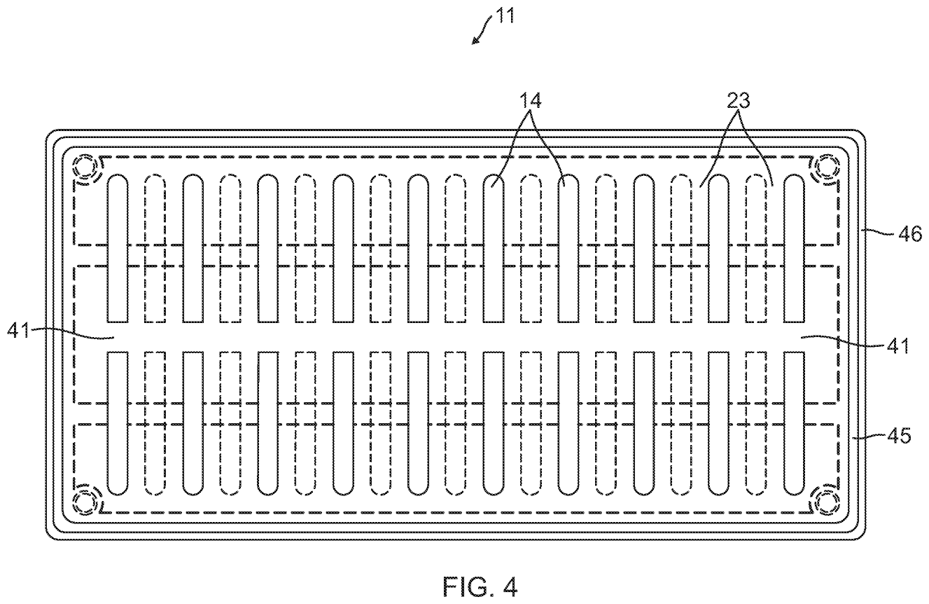

An alternative embodiment of device 11 is shown in FIGS. 4 and 5. Device 11 may be relatively rigid. When the ventilation device is made of materials which are somewhat flexible, such as rubber, it has been found that bridges 41 help maintain stability of lands 23 and, consequently, of slots 14 with respect to each other in plate 12. Similarly, bridges 42 span slots 43 in plate 13 for the same purpose.

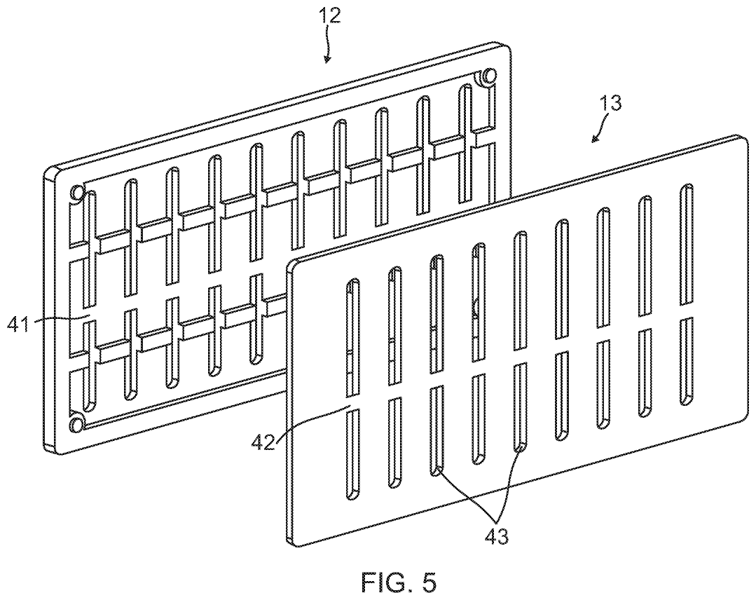

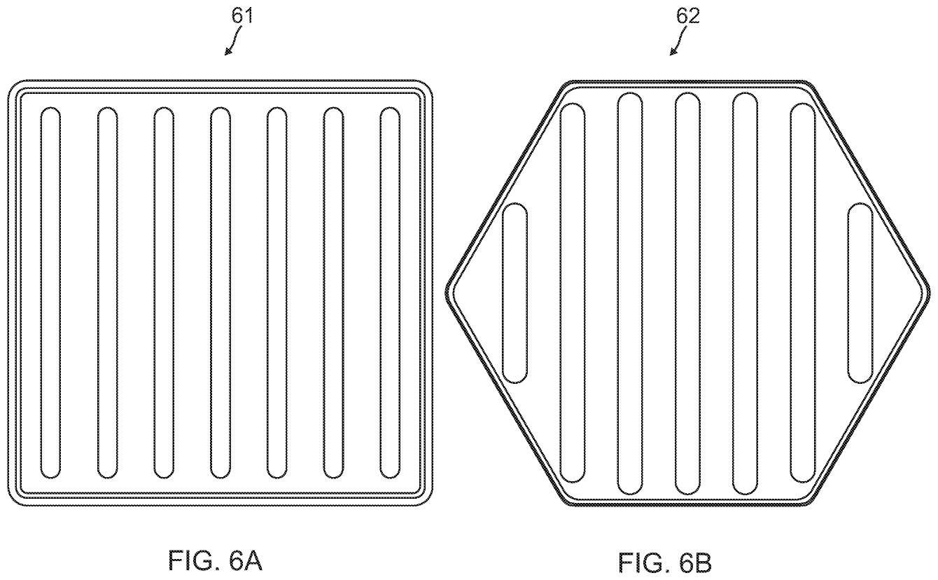

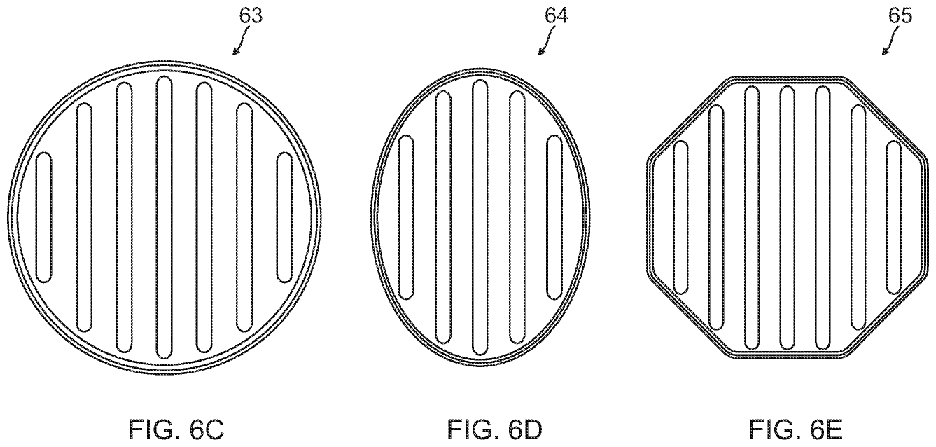

FIGS. 6A-E shows examples of possible shapes of ventilation device 11 in addition to the rectangular shape of FIG. 1. Each would include the same characteristics as the devices of FIGS. 1-5 so those will not be described in detail.

In FIG. 6A, ventilation device 61 is square, device 62 in FIG. 6B is hexagonal, device 63 in FIG. 6C is circular, device 64 in FIG. 6D is oval, and device 65 in FIG. 6E is octagonal. There may well be other possible or suitable shapes and those shown in the drawing are examples only.

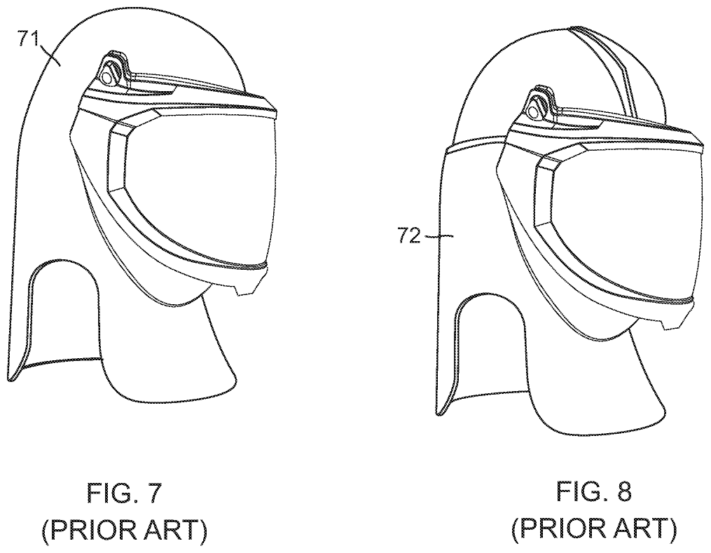

For reference purposes, FIG. 7 shows a typical hood 71 and protective face shield, and FIG. 8 shows a typical shroud 72 and a protective face shield, either of which may be used together with the ventilation device described herein. The only significant difference between the shroud of FIG. 8 is that it is attached to the helmet, whereas the hood of FIG. 7 is not attached to a helmet and fits over the user's head. Of course, a helmet should normally be worn by the user of the hood apparatus. Protective face shields are typically made of a polycarbonate with additives of organic dyes or nanoparticles, or a combination of both. Suitable fabrics from which protective hoods may be made are available from commercial sources. Typical acceptable fabrics include WESTEX, a registered trademark of Milliken & Company, and PROTERA and NOMEX MHP, registered trademarks of E.I. du Pont de Nemours and Company.

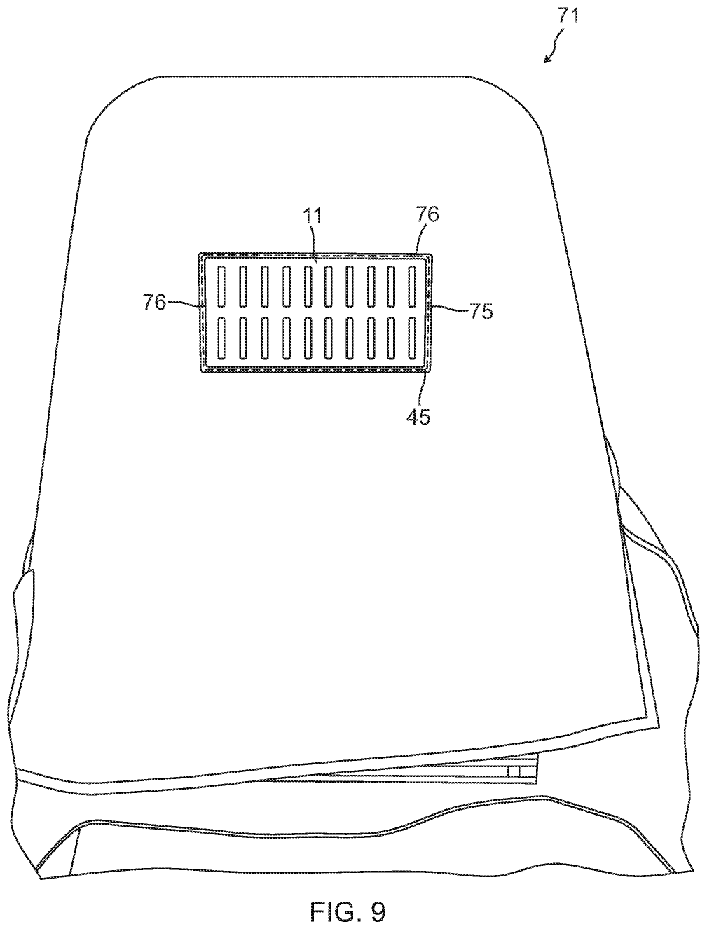

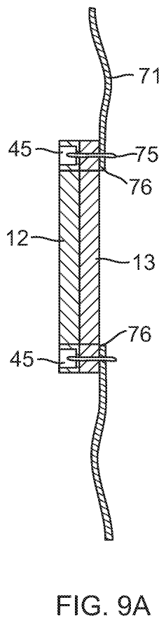

Given the situation when a worker is wearing a hood and protective face shield, as in FIGS. 7 and 8, plus a helmet, and is working in a potentially dangerous arc-energy area, heat may quickly build up within the hood. The preferred location for ventilation device 11 in hood 71, 72 is at the back of the neck as shown in FIG. 9, where the hood fabric would normally not be in contact with the user's skin and air can flow relatively freely. In this position the heated and possibly otherwise fouled air can escape, clear of any work area. Any location from the back of the user's head to the shoulders or below could provide the desired ventilation function.

To prepare the hood to receive ventilation device 11, a rectangular opening 76 is made in the hood in the area that would be at the back of the user's neck or in that general vicinity, and the unitary or assembled ventilation device 11 (FIGS. 1 and 4) is secured in the opening by suitable means. While it could be secured by an appropriate adhesive, for example, the preferred method is to stitch the device to the hood with appropriate fire retardant thread material 75 in groove 45 (see FIG. 1 ). Stitches of thread 75 can be seen in FIG. 9. Examples of materials from which thread 75 can be made include fiberglass, and KEVLAR and NOMEX, both registered trademarks of E.I. du Pont Nemours and Company for synthetic fibers and fabrics made from those fibers.

As can be seen in FIG. 9A, opening 76 in hood 71 is peripherally smaller than is ventilation device 11, enabling stitches 75 to positively secure the ventilation device in the opening.

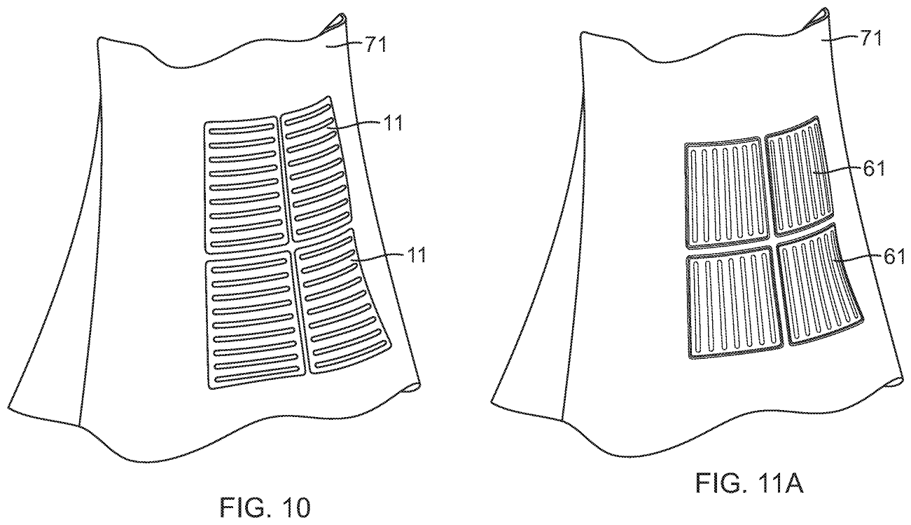

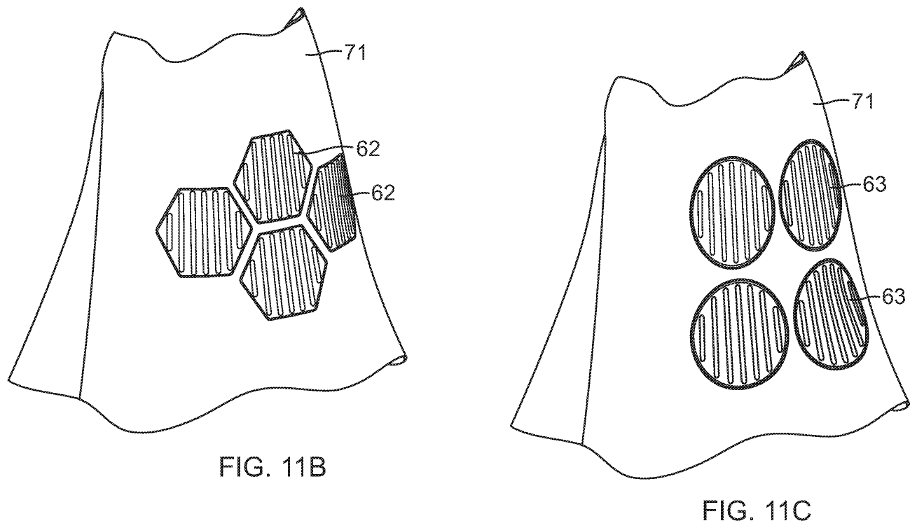

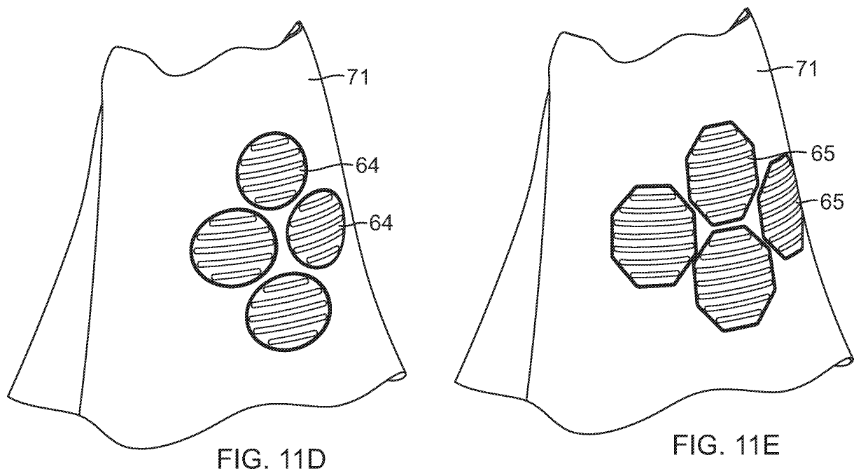

Some alternative hood/ventilation device arrangements are shown in FIGS. 10 and 11A-E. Hood segments 71 are employed in each figure with different ventilation device installations. For explanatory purposes, in FIG. 10 each hood segment is fitted with four ventilation devices. Depending upon the requirements of the purchaser, the number of ventilation devices could be as few as one to as many as size permits and requirements dictate.

In FIG. 10 there are four rectangular ventilation devices 11 arranged as shown. In this case the slots are oriented horizontally, but they could be oriented vertically as well.

Ventilation devices 61 are mounted to hood 71 in a square group of square ventilation devices in FIG. 11A. A group of bur hexagonal ventilation devices 62 are shown in FIG. 11B, a group of four circular devices 63 are mounted in hood 71 in FIG. 11C, four oval devices 64 are shown in FIG. 11D, and four octagonal devices 65 are shown in FIG. 11E.

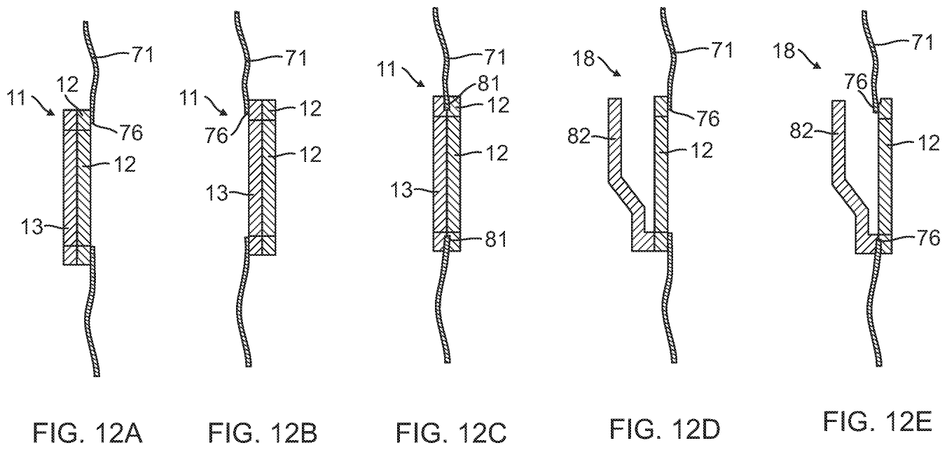

The manner of attachment, and some variations in the schematic partial cross-sectional representations of ventilation devices as they may be mounted to hood 71 are shown in FIGS. 12A-E.

FIG. 12A represents the mounting arrangement of FIG. 9, where device 11 is mounted on the inside of the fabric of hood 71. Alternatively, device 11 can be mounted over opening 76 on the outside of the hood fabric, as shown in FIG. 12B.

An alternative is shown in FIG. 12C, where device 11 is formed with a peripheral groove or slot 81 at the intersection of plates 12 and 13 so that the edges of opening 76 in the hood fabric are within that slot and the stitches are applied in the same way as before, through the hood fabric and through plates 12 and 13 in groove 45.

As another alternative mounting method, hood 71 can be two-ply and device 11 may be sandwiched between the hood fabric sheets, somewhat as a combination of FIGS. 12A and 12B.

In FIGS. 12D and 12E the ventilation device 18 has the usual slotted plate 12 connected to inside baffle plate 82 instead of flat slotted back plate 13. The same criteria apply as before, that is, that there is no direct path through the slots in plate 12 that can get past element 82 so any arc-flash energy cannot get through hood 71. Any energy that enters through the slots in plate 12 are blocked by baffle plate 82. With this configuration element 82 does not have any slots at all because air is ventilated through the space between plate 12 and element 82. Any arc-flash energy that impinges upon front plate 12 is partially blocked and absorbed by plate 12, and any such energy that passes through slots in plate 12 are absorbed and deflected by baffle plate 82. Even though there are no slots in plate 82, the flow through plate 12 to plate 82 is still in a semi-tortuous path.

Plate 12 in FIG. 12D is mounted in opening 76 in hood 71 in the same manner as shown in FIG. 12A. Plates 12 and 82 in FIG. 12E are mounted to hood 71 as shown in FIG. 12B at the top and as shown in FIG. 12C at the bottom where edge 76 of the hood opening is in a groove at the intersection of the two plates.

The alternative configurations and mounting arrangements of FIG. 12 are provided to show that there may be many ways that the ventilation device may be mounted in an opening 76 in the fabric of hood 71, and these are merely examples to suggest that there may be many more.

While the ventilation device described here would likely provide adequate ventilation under many environments, it could also be used in conjunction with a fan system for enhanced ventilation.

1. A device for use with a safety face shield assembly, the device comprising:
a hood configured to cover at least the head of a wearer and incorporating the safety face shield, the hood and safety face shield being formed of materials that protect the wearer from arc-flashes, said hood having an opening in at least one location;
a ventilation device mounted in said opening, said ventilation device having a periphery, said hood being secured to the periphery of said ventilation device, said ventilation device comprising:
a first plate having a first plurality of slots therethrough; and
a second plate having a second plurality of slots therethrough;
said first and second plates being arranged in face-to-face relationship with said slots in said first and second plates being in offset registration so that there is no direct fluid flow route through the slots in said front and back plates, thereby providing a semi-tortuous path for ventilation between internal and external sides of said hood, said ventilation device being formed of materials that block arc-flashes, the semi-tortuous path thereby protecting the wearer from an external arc-flash.

2. The device recited in claim 1, wherein said first plurality of slots differs in number from said second plurality of slots.

3. The device recited in claim 1, wherein said first plurality of slots is equal in number to said second plurality of slots.

4. The device recited in claim 1, wherein said first plate is formed with a peripheral rim that is thicker than B central portion of said first plate which central portion faces said second plate, thereby permitting air flow in a semi-tortuous path from the slots in one of said first and second plates to and through the slots in the other of said first and second plates.

5. The device recited in claim 4, wherein said central portion of said first plate is formed with a plurality of stand-offs which are in the plane of said peripheral rim.

6. The device recited in claim 1, wherein said first plurality of slots defines a first plurality of lands therebetween, and said second plurality of slots defines a second plurality of lands therebetween.

7. The device recited in claim 5, wherein said first plurality of slots defines a first plurality of lands therebetween, and said second plurality of slots defines a second plurality of lands therebetween and wherein said plurality of stand-offs are formed on said first plurality of lands to maintain spacing between the facing surfaces of said first and second plates.

8. The device recited in claim 6, and further comprising a first plurality of bridges in said first plurality of slots between each two of said lands defining each said slot, said first plurality of bridges connecting adjacent lands of said first plurality of lands.

9. The device recited in claim 6, and further comprising a second plurality of bridges in said second plurality of slots between each two of said lands defining each said slot, said second plurality of bridges connecting adjacent lands of said second plurality of lands.

10. The device recited in claim 1, wherein said first plate is formed with a peripheral groove in the surface facing away from said second plate.

11. The device recited in claim 10, wherein said ventilation device is mounted in said opening by stitches through said peripheral groove in said first plate, through said second plate, and through the hood material.

12. A ventilation device for use in a hood made of arc-flash protective material and having at least one opening therethrough, said ventilation device having a periphery adapted to be secured in the opening in said hood, said ventilation device comprising:
a first plate having a first plurality of slots therethrough; and
a second plate having a second plurality of slots therethrough;
said first and second plates being arranged in face-to-face relationship with said slots in said first and second plates being in offset registration so that there is no direct fluid flow route through the slots in said first and second plates, thereby providing a semi-tortuous path for ventilation between internal and external sides of said hood, said ventilation device being formed of materials that block arc-flashes, the semi-tortuous path thereby protecting the wearer from an external arc-flash.

13. The ventilation device recited in claim 12, wherein said first plurality of slots differs in number from said second plurality of slots.

14. The ventilation device recited in claim 12, wherein said first plurality of slots is equal in number to said second plurality of slots.

15. The ventilation device recited in claim 12, wherein said first plate is formed with a peripheral rim that is thicker than a central portion of said first plate which central portion faces said second plate, thereby permitting air flow in a semi-tortuous path from the slots in one of said first and second plates to and through the slots in the other of said first and second plates.

16. The ventilation device recited in claim 15, wherein said central portion of said first plate is formed with a plurality of stand-offs which are substantially equal in thickness to said peripheral rim.

17. The ventilation device recited in claim 12, wherein said first plate is comprised of at least two coplanar segments.

18. The ventilation device recited in claim 12, wherein said second plate is comprised of at least two coplanar segments.

19. The ventilation device recited in claim 12, wherein said first plate is formed with a peripheral groove in the surface facing away from said second plate.

20. The ventilation device recited in claim 19, wherein said ventilation device is securable in said opening in said hood by stitches through said peripheral groove in said first plate, through said second plate, and through the hood protective material.

21. A device for use with a safety face shield assembly, the device comprising:
a hood configured to cover at least the head of a wearer and incorporating the safety face shield, the hood and safety face shield being formed of materials that protect the wearer from arc-flashes, said hood having an opening in at least one location;
a ventilation device mounted in said opening, said ventilation device having a periphery, said hood being secured to the periphery of said ventilation device, said ventilation device comprising:
a first plate having a first plurality of slots therethrough; and
a baffle plate mounted to and at least partially spaced from said first plate;
said first and baffle plates being arranged in face-to-face relationship so that there is no direct fluid flow route through the slots in said first plate and past said baffle plate, thereby providing a semi-tortuous path for ventilation between internal and external sides of said hood, said ventilation device being formed of materials that block arc-flashes, the semi-tortuous path thereby protecting the wearer from an arc-flash.

22. The device recited in claim 21, wherein said first plate is formed with an inside surface and an outside surface and a peripheral groove in one said surface.

23. The device recited in claim 22, wherein said ventilation device is mounted in said opening by stitches through said peripheral groove in said from plate and through said hood protective material.

24. A ventilation device for use in a hood made of arc-flash protective material and having at least one opening therethrough, said ventilation device having a periphery adapted to be secured in the opening in said hood, said ventilation device comprising:
a first plate having a first plurality of slots therethrough; and
a baffle plate mounted to and at least partially spaced from said first plate;
said first and baffle plates being arranged in face-to-face relationship so that there is no direct fluid flow route through the slots in said first plate and past said baffle plate, thereby providing a semi-tortuous path for ventilation between internal and external sides of said hood, said ventilation device being formed of materials that block arc-flashes, the semi-tortuous path thereby protecting the wearer from an arc-flash.

25. The ventilation device recited in claim 24, wherein said first plate is formed with an inside surface and an outside surface and a peripheral groove in one said surface.

26. The ventilation device recital in claim 25, wherein said ventilation device is securable in said opening in said hood by stitches through said peripheral groove in said front plate and through said hood protective material.
